(19) United States
(12) Patent Application Publication
(10) Pub. No.: US 2026/0047848 A1
(43) Pub. Date: Feb. 19, 2026

(54) TAMPONADE DEVICE

(71) Applicant: Myodynamics, LLC, Englewood Cliffs, NJ (US)

(72) Inventors: Sreejit Nair, Pembroke Pines, FL (US); Ashwani Sastry, Orlando, FL (US)

(21) Appl. No.: 18/805,278

(22) Filed: Aug. 14, 2024

(51) Int. Cl. A61B 17/12

(52) U.S. Cl. CPC A61B 17/12159; A61B 17/1204; A61B 17/12109; A61B 2017/12004; A61B 2017/12054

(57) ABSTRACT

The present disclosure relates to a tamponade device. The tamponade device includes a sheath member. The sheath member includes a hollow cavity defined along a length of the sheath member. The hollow cavity is configured to allow the insertion of a guidewire therein. The sheath member is configured to be positioned at a vascular access point of a subject over a guidewire deployed into the lumen of a blood vessel at the vascular access site. Further, the sheath member is configured with a predefined diametrical size that impedes the tamponade device from being deployed into the lumen of the blood vessel through the vascular access point, thereby positioning the sheath member at the vascular access point to occlude the vascular access point. By applying focal pressure or compression to the blood vessel directly at the vascular access site, hemostasis can be achieved using the tamponade device.

### TECHNICAL FIELD

The present disclosure relates to medical devices and more particularly relates to a tamponade device to occlude an entry site of a blood vessel to stop bleeding.

### BACKGROUND

Endovascular surgery is a field of medicine where a physician makes use of minimizing invasive techniques to treat arteries and veins within a body (e.g., human body). The arteries and veins can be in the peripheral vasculature (legs, arms, abdomen), cerebral vasculature, or cardiac vasculature (e.g., coronary arteries, coronary veins). The instruments (e.g., catheters, introducer sheaths, angioplasty balloons, stents, guidewire, etc.) are manually inserted into the body and can be manipulated, for example, by advancing, retracting, rotating clockwise, and rotating counterclockwise, by the physician. Endovascular procedures become more mainstream and more complex, allowing more pathologies to be treated in a minimally invasive fashion/manner.

Further, there is an occurrence of bleeding due to endovascular procedures for various reasons ranging from oozing to severe hemorrhage. To prevent or stop bleeding (i.e., hemostasis) various techniques and interventions are performed by trained physicians. Traditionally, hemostasis is achieved by manually applying pressure to the puncture site to compress the blood vessel and promote clot formation at the entry site. However, the traditional manual pressure method of hemostasis is associated with prolonged compression time, causes discomfort to the patient and/or operators, can have inconsistent pressure application, and thereby reduce the efficiency and success of achieving hemostasis.

In recent times, Vascular Closure Devices (VCDs) have been used to achieve hemostasis (stoppage of bleeding) and facilitate closure of arterial access sites following minimally invasive vascular procedures, such as cardiac catheterization, angiography, or percutaneous vascular interventions. However, the use of the vascular closure device requires appropriate training and expertise to minimize the risk of complications, such as bleeding, hematoma formation, pseudoaneurysm, or vascular injury. Therefore, the selection and deployment of the vascular closure device should be guided by clinical judgment expertise, patient characteristics, and procedural considerations. There are some drawbacks to the existing vascular closure devices that are available today. In some cases, the vascular closure device maybe complicated to operate leading to a high learning curve and possible user error. In other cases, a vascular closure device might fail to achieve effective hemostasis or closure of the access site even when used properly, leading to prolonged bleeding, inadequate sealing. Furthermore, there could be complications resulting from the use of a given vascular closure device that might result in the need for additional interventions or surgery. Lastly, the existing vascular closure devices are expensive.

Therefore, there is a need for an efficient tamponade device (or a vascular closure device) to achieve hemostasis to overcome one or more limitations stated above, in addition to providing other technical advantages.

### SUMMARY

Various embodiments of the present disclosure provide a tamponade device to occlude the entry site into a blood vessel and apply pressure at a vascular access point of a subject.

In an embodiment, the tamponade device is disclosed. The tamponade device includes a sheath member. The sheath member includes a hollow cavity defined along a length of the sheath member. The hollow cavity is configured to allow insertion of a guidewire therein. The sheath member is configured to be positioned at a vascular access point of a subject over the guidewire deployed in a lumen of a blood vessel. Further, the sheath member is configured with a predefined diametrical size that impedes the tamponade device from being deployed into the lumen of the blood vessel at the vascular access point of the subject; this allows the sheath member to be positioned at the vascular access point to occlude the vascular access point of the blood vessel.

In another embodiment, a tamponade device to occlude the entry site into a blood vessel is disclosed. The tamponade device includes a sheath member. The sheath member includes a hollow cavity defined along a length of the sheath member. The hollow cavity is configured to allow the insertion of a guidewire therein. The sheath member is configured to be positioned at a vascular access point of a subject over the guidewire deployed in a lumen of the blood vessel. Further, the sheath member is configured with a predefined diametrical size that impedes the tamponade device from being deployed into the lumen of the blood vessel at the vascular access point of the subject, thereby positioning the sheath member at the vascular access point to occlude the vascular access point of the blood vessel. Through the hollow cavity of the sheath member, a plunger is inserted to plug the cavity as well as the arteriotomy and venotomy.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device or a tool and instrumentalities disclosed herein. Moreover, those in the art will understand that the drawings are not to scale. Wherever possible, like elements have been indicated by identical numbers:

FIG. 1A illustrates a schematic view of a subject under endovascular procedures, in accordance with an embodiment of the present disclosure;

FIG. 1B illustrates a schematic view of a tamponade device being inserted into the subject, in accordance with an embodiment of the present disclosure;

FIG. 2A illustrates a perspective view of a tamponade device, in accordance with an embodiment of the present disclosure;

FIG. 2B illustrates a cross-sectional view of the tamponade device along a cross-sectional axis A-A′ of the tamponade device of FIG. 2A, in accordance with an embodiment of the present disclosure;

FIG. 2C illustrates a perspective view of the tamponade device of FIG. 2A accommodating a guidewire, in accordance with an embodiment of the present disclosure;

FIGS. 3A and 3B illustrate a schematic representation of the tamponade device of FIG. 2A used for occluding a blood vessel, in accordance with another embodiment of the present disclosure;

FIG. 4A illustrates a schematic representation of a tamponade device, in accordance with an embodiment of the present disclosure;

FIG. 4B illustrates a cross-sectional view of the tamponade device along a cross-sectional axis B-B′ of the tamponade device of FIG. 4A, in accordance with an embodiment of the present disclosure;

FIG. 4C illustrates a perspective view of the tamponade device of FIG. 4A accommodating a guidewire, in accordance with an embodiment of the present disclosure;

FIGS. 5A and 5B illustrate a schematic representation of the tamponade device of FIG. 4A used for occluding the blood vessel, in accordance with another embodiment of the present disclosure; and

FIG. 6 illustrates a schematic representation of a dilator device, in accordance with one example embodiment of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

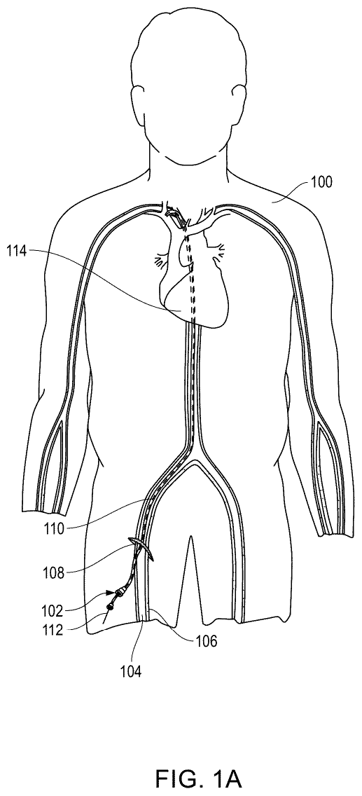

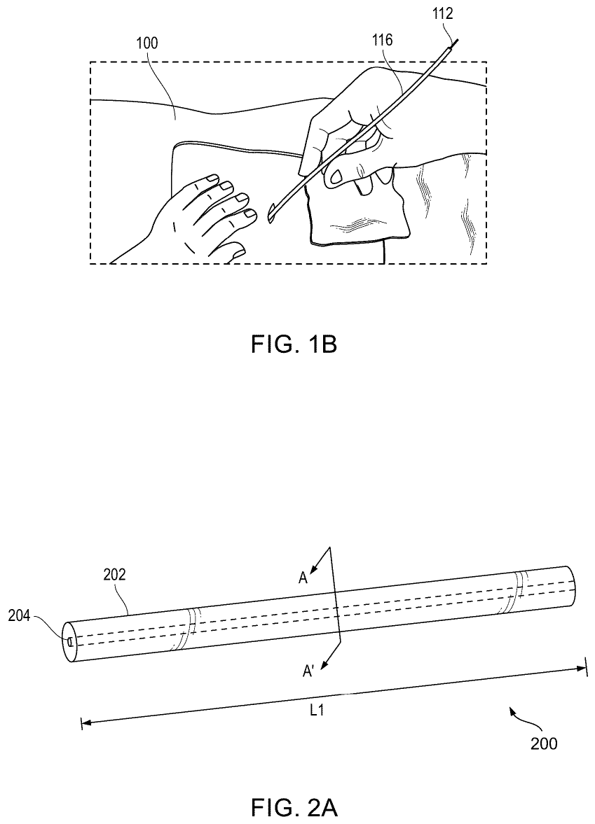

Various embodiments of the present disclosure are described with reference to FIGS. 1A and 1B to FIG. 6.

FIG. 1 A illustrates a schematic view of a subject 100 under endovascular procedures, in accordance with an example embodiment of the present disclosure. As shown, a medical device 102 is deployed into a lumen 104 of a blood vessel 106 through a vascular access point 108 of the subject 100. As an example, the medical device 102 is deployed in the blood vessel 106 (e.g., a femoral artery). The femoral artery is a common access site for performing an endovascular procedure such as cardiac catheterization. More specifically, the medical device 102 inserted into the lumen 104 of the blood vessel 106 provides access to an internal organ of the subject 100.

The medical device 102 includes an intravascular sheath 110 and a guidewire 112. The intravascular sheath 110 is a thin flexible tube adapted to be inserted into the blood vessel 106 to permit the introduction or withdrawal of fluids or to keep the vascular access point 108 open. Typically, the intravascular sheath 110 is deployed at a target site (or an operating site) of the subject 100 using the guidewire 112. The size and location of the vascular access point 108 used to access the blood vessel 106 may vary based on the procedures involved. In an example, when treating the coronary vasculature of the heart 114, the vascular access point 108 may be the right groin, with the medical device 102 passing into the right coronary artery (as shown in FIG. 1A).In another example, in the case of performing an angiography (i.e., medical imaging technique in the heart 114), the guidewire 112 and the intervascular sheath 110 are inserted in the coronary artery and chambers of the of the heart 114.

After performing the endovascular procedure (i.e., angiography), the physician retracts the intervascular sheath 110 (or the medical device 100) from the subject 100. It is to be noted that the guidewire 112 is retracted from the target area and retained within the lumen 104 (i.e., just below the vascular access point 108). To achieve hemostasis at the vascular access point 108, a tamponade device 116 may be used.

Referring to FIG. 1B,a schematic view of the tamponade device 116 being inserted into the subject 100 is shown, in accordance with an example embodiment of the present disclosure. The tamponade device 116 is used to apply focal pressure at the vascular access point 108 to partially or completely occlude bleeding post endovascular procedures.

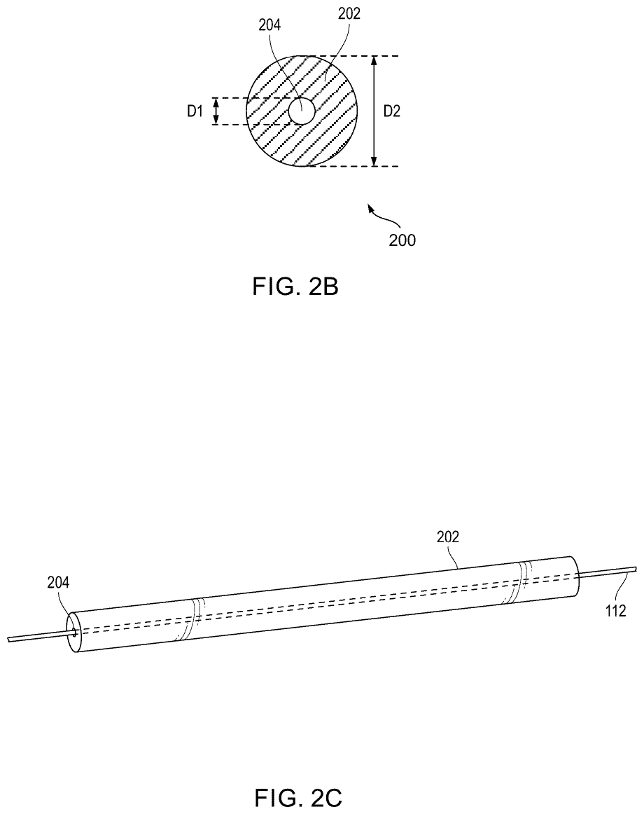

FIG. 2A illustrates a perspective view of a tamponade device 200, in accordance with one embodiment of the present disclosure. The tamponade device 200 is an example of the tamponade device 116 of FIG. 1B.The tamponade device 200 includes a sheath member 202, for example, a solid sheath member. The sheath member 202 may be an elongated tube. The sheath member 202 is configured to be a cylindrical structure. Alternatively, the sheath member 202 may be configured in any other structural configurations compatible with the blood vessel 106 or as per the requirements.

The sheath member 202 includes a hollow cavity 204 (e.g., small hollow cavity) defined along a length (L1) of the sheath member 202. The hollow cavity 204 is configured with a first dimension. The first dimension of the hollow cavity 204 corresponds to an inner diameter (see, ‘D1’ of FIG. 2B) of the sheath member 202. The hollow cavity 204 of the first dimension allows insertion of the guidewire 112 therein. It is to be noted that the inner diameter DI defined by the hollow cavity 204 is configured in conformity to the guidewire 112 for allowing only the guidewire 112 to be inserted there through (as shown in FIG. 2C). This allows the sheath member 202 to be positioned at the vascular access point 108 (as shown in FIG. 1A) through the guidewire 112.

Further, the sheath member 202 is made of plastic materials such as biocompatible plastic materials. The biocompatible plastic materials meet stringent requirements to ensure patient safety and regulatory compliance. Some non-limiting examples of the biocompatible plastic materials used in the sheath member 202 may be silicone, Polyvinyl chloride (PVC), Polyethylene (PE), Polycarbonate (PC), Polyethylene terephthalate (PET), Polytetrafluoroethylene (PTFE), and the like.

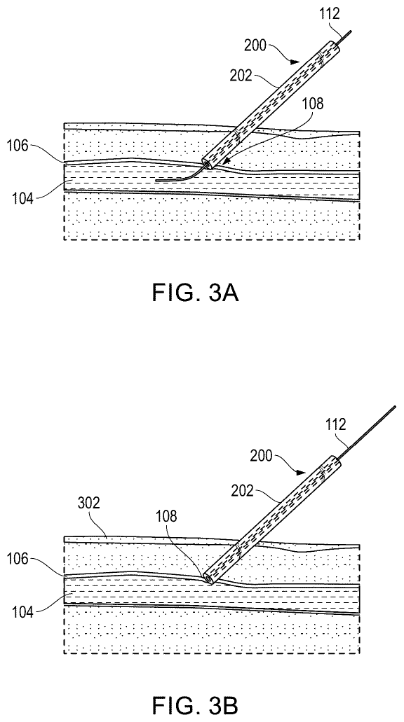

Referring to FIG. 3A, after performing the endovascular procedure, the intravascular sheath 110 is exchanged with the tamponade device 200. At this stage, the guidewire 112 is still retained inside the lumen 104 of the blood vessel 106. More specifically, the sheath member 202 is configured to be positioned at the vascular access point 108 of the subject 100 through the guidewire 112 deployed in the lumen 104 of the blood vessel 106. The sheath member 202 is configured with a predefined diametrical size. The predefined diametrical size corresponds to an outer diameter (see, ‘D2’ of FIG. 2B) of the sheath member 202. The outer diameter D2 of the sheath member 202 is configured to be greater than a size (i.e. circumference) of the vascular access point 108. In other words, a larger French size of the tamponade device 200 may be greater than the arteriotomy/venotomy (i.e., the blood vessel 106 or the vascular access point 108).Hence, the sheath member 202 inserted over the guidewire 112 is prevented from entering the lumen 104 of the blood vessel 106. In other words, the tamponade device 200 is prevented (or impeded) from being deployed into the lumen 104 of the blood vessel 106 against the vascular access point 108 of the subject 100. As a result, the sheath member 202 is positioned (or disposed) at the vascular access point 108 to occlude (or seal) the vascular access point 108 of the blood vessel 106. For example, the sheath member 202 may be configured with a French size 3 Fr larger than the size of the arteriotomy to occlude the vascular access point 108.

Referring to FIG. 3B, the sheath member 202 positioned at the vascular access point 108 is configured to apply focal pressure at one of a skin level (see, 302) of the subject 100 and the vascular access point 108 of the subject 100. This results in achieving hemostasis at the vascular access point 108. Specifically, the focal pressure is applied at the vascular access point 108 while the guidewire 112 is retracted from the lumen 104 and kept above the vascular access point 108. To prevent bleeding from the vascular access point 108, the pressure is applied by the sheath member 202 at the vascular access point 108. In other words, the pressure applied by the sheath member 202 at the vascular access point 108 temporarily holds the blood flow out from the vascular access point 108 with the retracted guidewire 112 further sealing the vascular access point 108 (i.e., arteriotomy site or arteriotomy). It is to be noted that the pressure is applied via the sheath member 202 until the hemostasis is achieved in the vascular access point 108.

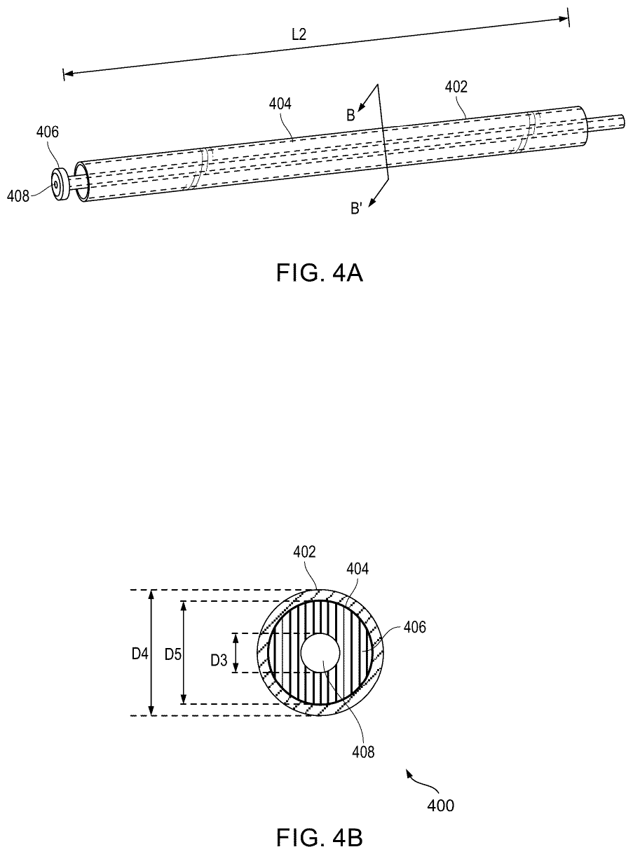

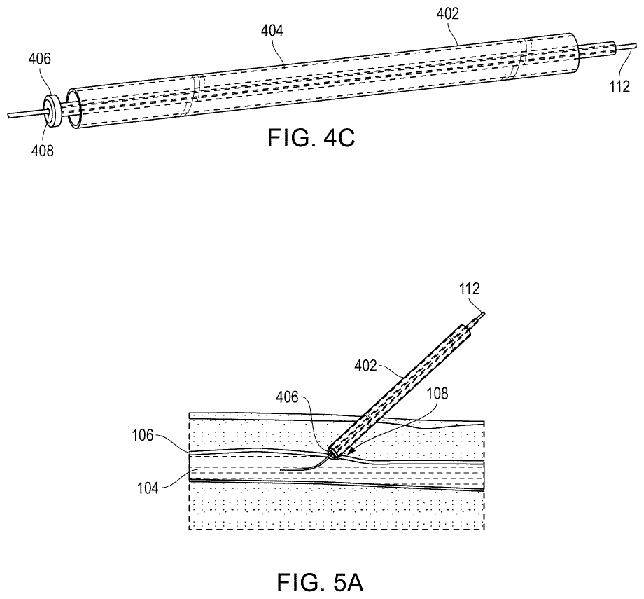

FIG. 4A illustrates a schematic representation of a tamponade device 400, in accordance with an embodiment of the present disclosure. The tamponade device 400 is an example of the tamponade device 116 of FIG. 1B. The tamponade device 400 includes a sheath member 402. The sheath member 202 may be an elongated tube. In one embodiment, the sheath member 402 includes a hollow cavity 404 defined along a length (L2) of the sheath member 402. The hollow cavity 404 is configured with a second dimension. The second dimension of the hollow cavity 404 corresponds to an inner diameter (see, ‘D3’ of FIG. 4B). The sheath member 402 is configured to be cylindrical structure. Alternatively, the sheath member 402 may be configured in any other structural configurations compatible with the blood vessel 106 or as per the requirements.

Further, the tamponade device 400 includes a plunger 406. The plunger 406 is configured with an outer circumferential surface in conformity with the second dimension (or the inner diameter ‘D3’ of the hollow cavity 404 of the sheath member 402. This allows the plunger 406 to be accommodated in the hollow cavity 404 of the sheath member 402. In other words, the plunger 406 is configured to be inserted in the hollow cavity 404 and slidable along the length L2 of the sheath member 402. The plunger 406 may include a passageway 408 for allowing insertion of the guidewire 112 therein (as shown in FIG. 4C). The sheath member 402 and the plunger 406 accommodated therein are positioned at the vascular access point 108 of the subject 100 through the guidewire 112 deployed in the lumen 104 of a blood vessel 106. Further, the sheath member 402 adapted to guide the plunger 406 to be positioned at the vascular access point 108 conforms to a guide sheath member. Furthermore, the tamponade device 400 including the guide sheath member (or the sheath member 402) and the plunger 406 may be referred to as ‘a sheath tamping device’. The plunger 406 provides solid support to the sheath member 402 for applying focal pressure at the vascular access point 108 which will be explained further in detail.

Further, the sheath member 402 is made of plastic materials such as biocompatible plastic materials. The biocompatible plastic materials meet stringent requirements to ensure patient safety and regulatory compliance. Some non-limiting examples of the biocompatible plastic materials used in the sheath member 402 may be silicone, Polyvinyl chloride (PVC), Polyethylene (PE), Polycarbonate (PC), Polyethylene terephthalate (PET), Polytetrafluoroethylene (PTFE), and the like.

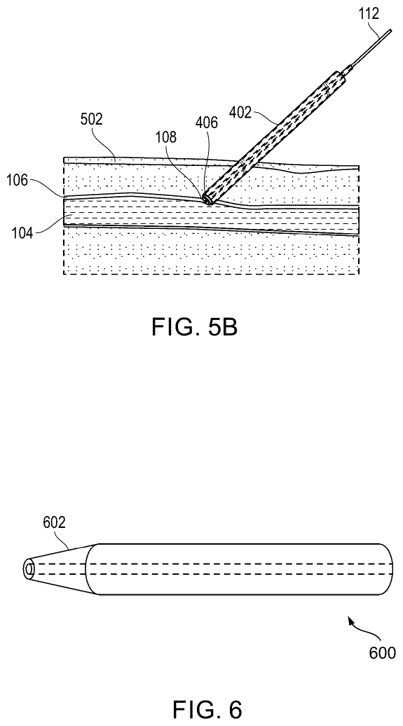

Referring to FIG. 5A, after performing the endovascular procedure, the intravascular sheath 110 is exchanged with the tamponade device 400. At this stage, the guidewire 112 is still retained inside the lumen 104 of the blood vessel 106. More specifically, the sheath member 402 is configured to be positioned at the vascular access point 108 of the subject 100 through the guidewire 112 deployed in the lumen 104 of the blood vessel 106. The sheath member 402 is configured with a predefined diametrical size. The predefined diametrical size corresponds to an outer diameter (see, ‘D4’ of FIG. 4B) of the sheath member 402. Further, the plunger 406 is configured with an outer circumference (or an outer diameter'D5′of FIG. 4B). Hence, the predefined diametrical size of the sheath member 402 and the outer circumference of the plunger 406 impedes the tamponade device 400 from being deployed into the lumen 104 of the blood vessel 106 against the vascular access point 108 of the subject 100. To that effect, the sheath member 402 and the plunger 406 are positioned at the vascular access point 108 to occlude the vascular access point 108 of the blood vessel 106. For example, the sheath member 402 and the plunger 406 may be configured with a French size 3 Fr larger than the size of the arteriotomy to occlude the vascular access point 108.

Referring to FIG. 5B, the sheath member 402 and the plunger 406 positioned at the vascular access point 108 are configured to apply focal pressure at one of a skin level (see, 502) of the subject 100 and the vascular access point 108 of the subject 100. This results in achieving hemostasis at the vascular access point 108. Specifically, the focal pressure is applied at the vascular access point 108 while the guidewire 112 is retracted from the lumen 104 and kept above the vascular access point 108. To prevent bleeding from the vascular access point 108, the pressure is applied by the sheath member 402 and the plunger 406 at the vascular access point 108. In other words, the pressure applied by the sheath member 402 and the plunger 406 at the vascular access point 108 temporarily holds the blood flow out from the vascular access point 108 and blocks the bleeding due to removal of the guidewire 112. It is to be noted that the pressure is applied via the sheath member 402 and the plunger 406 until the hemostasis is achieved at the vascular access point 108.

It should be noted that the focal pressure applied at the vascular access point 108 by the tamponade device 200 or the tamponade device 400 may be a manual pressure applied by a physician or may be through an external pressure device coupled to the tamponade device 200 or the tamponade device 400. As explained above, the pressure is applied by the tamponade device 200 or the tamponade device 400 until the hemostasis is achieved at the vascular access point 108. Generally, the pressure is applied for a predefined time (e.g., 15 minutes) at the vascular access point 108 to achieve hemostasis. In other words, the tamponade devices 200/400/116 may apply pressure at the vascular access point 108 for about 15 minutes to achieve hemostasis at the vascular access point 108. In an example, the vascular access point 108 may be an arteriotomy site or venotomy site. Thus, the tamponade device may be used to achieve hemostasis by applying focal pressure/compression to the blood vessel directly at the vascular access site.

In an embodiment, the tamponade devices 116/200/400 may include an imaging device (e.g., ultrasound) to assist the physician in operating the tamponade devices 116/200/400 to seal or occlude the vascular access point 108.

In an example scenario, the tamponade devices 116/200/400 may not be able to seal/occlude the vascular access point 108 due to dimension constraints. In other words, there may be difficulty in introducing the tamponade devices 116/200/400 to the point of the vascular access point 108 (e.g., arteriotomy site). In this scenario, the tamponade devices 116/200/400 may be configured to couple with a dilator device (see, 600 of FIG. 6). The dilator device 600 may be configured with functionality similar to the plunger 406. The dilator device 600 may be configured with a tapered end (see, 602 of FIG. 6). The dilator device 600 may be configured to induce a serial dilation at the vascular access point 108. Thereafter, the tamponade devices 116/200/400 may be positioned at the vascular access point 108 through the guidewire 112 as explained above. Further, depending on the of the tamponade devices 116/200/400, the size of the dilator device 600 used may vary. In one embodiment, the dilator device 600 of size, e.g., 7 mm, is used for dilating the passage to the vascular access point 108 of size, e.g., 3 mm. In some embodiments, the dilator device 600 of different sizes (e.g., 5 mm, 6 mm, and 7 mm) may be used for dilating the passage to the vascular access point 108 of size (e.g., 3 mm). The dilation process facilitates easy insertion of the tamponade devices 116/200/400 into the subject 100.

Various embodiments of the disclosure, as discussed above, may be practiced with steps and/or operations in a different order, and/or with hardware elements in configurations, which are different than those which, are disclosed. Therefore, although the disclosure has been described based upon these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A tamponade device, comprising:
a sheath member comprising a hollow cavity defined along a length of the sheath member, the hollow cavity configured to allow insertion of a guidewire therein,
wherein the sheath member is configured to be positioned at a vascular access point of a subject over the guidewire deployed in a lumen of a blood vessel, and
wherein the sheath member is configured with a predefined diametrical size that impedes the tamponade device from being deployed into the lumen of the blood vessel through the vascular access point of the subject, thereby positioning the sheath member at the vascular access point to occlude the vascular access point of the blood vessel.

2. The tamponade device as claimed in claim 1, wherein the predefined diametrical size corresponds to an outer diameter of the sheath member, and wherein the outer diameter of the sheath member is greater than a size of the vascular access point, thereby impeding the tamponade device from being deployed into the lumen of the blood vessel through the vascular access point.

3. The tamponade device as claimed in claim 1, wherein the hollow cavity is configured with a first dimension, the first dimension of the hollow cavity corresponding to an inner diameter of the sheath member, and wherein the hollow cavity of the first dimension allows insertion of the guidewire therein.

4. The tamponade device as claimed in claim 1, wherein the sheath member positioned at the vascular access point is configured to apply focal pressure at one of a skin level of the subject and the vascular access point of the subject, thus resulting in hemostasis at the vascular access point.

5. The tamponade device as claimed in claim 1, further comprising:
a plunger comprising a passageway for allowing the guidewire to be inserted therein.

6. The tamponade device as claimed in claim 5, wherein an outer circumferential surface of the plunger is configured in conformity with a second dimension of a hollow cavity of a sheath member, thereby allowing the plunger to be accommodated in the hollow cavity of the sheath member.

7. The tamponade device as claimed in claim 6, wherein the second dimension of the hollow cavity corresponds to an inner diameter of the sheath member.

8. The tamponade device as claimed in claim 6, wherein the sheath member and the plunger accommodated therein are positioned at a vascular access point of a subject over a guidewire deployed in a lumen of a blood vessel.

9. The tamponade device as claimed in claim 8, wherein the sheath member of a predefined diametrical size and the plunger of an outer circumference impedes the tamponade device from being deployed into the lumen of the blood vessel through the vascular access point of the subject, thereby positioning the sheath member and the plunger at the vascular access point to occlude the vascular access point of the blood vessel.

10. The tamponade device as claimed in claim 9, wherein the sheath member and the plunger positioned at the vascular access point are configured to apply focal pressure at one of a skin level of the subject and the vascular access point of the subject, thus resulting in hemostasis at the vascular access point.

11. The tamponade device as claimed in claim 10, wherein the sheath member adapted to guide the plunger to be positioned at the vascular access point conforms to a guide sheath member.

12. The tamponade device as claimed in claim 1, wherein the sheath member is made of biocompatible plastic materials.

13. The tamponade device as claimed in claim 1, wherein the sheath member is positioned at the vascular access point to occlude the lumen of the blood vessel based on a serial dilation induced by a dilator device at the vascular access point.

14. A tamponade device to occlude a blood vessel, comprising:
a sheath member comprising a hollow cavity defined along a length of the sheath member, the hollow cavity configured to allow insertion of a guidewire therein,
wherein the sheath member is configured to be positioned at a vascular access point of a subject over the guidewire deployed in a lumen of the blood vessel,
wherein the sheath member is configured with a predefined diametrical size that impedes the tamponade device from being deployed into the lumen of the blood vessel through the vascular access point of the subject, thereby positioning the sheath member at the vascular access point to occlude the vascular access point of the blood vessel, and
wherein the predefined diametrical size corresponds to an outer diameter of the sheath member, and wherein the outer diameter of the sheath member is greater than a size of the vascular access point, thereby preventing the tamponade device from being deployed into the lumen of the blood vessel through the vascular access point.

15. The tamponade device as claimed in claim 14, wherein the sheath member positioned at the vascular access point is configured to apply focal pressure at one of a skin level of the subject and the vascular access point of the subject, thus resulting in hemostasis at the vascular access point.

16. The tamponade device as claimed in claim 14, wherein the hollow cavity is configured with a first dimension, the first dimension of the hollow cavity corresponding to an inner diameter of the sheath member, and wherein the hollow cavity of the first dimension allows insertion of the guidewire therein.

17. The tamponade device as claimed in claim 14, further comprising:
a plunger comprising a passageway for allowing the guidewire to be inserted therein, wherein an outer circumferential surface of the plunger is configured in conformity with a second dimension of a hollow cavity of a sheath member, thereby allowing the plunger to be accommodated in the hollow cavity of the sheath member.

18. The tamponade device as claimed in claim 17, wherein the second dimension of the hollow cavity corresponds to an inner diameter of the sheath member, and wherein the sheath member and the plunger accommodated therein are positioned at a vascular access point of a subject over a guidewire deployed in a lumen of a blood vessel.

19. The tamponade device as claimed in claim 18, wherein the sheath member of a predefined diametrical size and the plunger of an outer circumference impedes the tamponade device from being deployed into the lumen of the blood vessel through the vascular access point of the subject, thereby positioning the sheath member and the plunger at the vascular access point to occlude the vascular access point of the blood vessel, and
wherein the sheath member and the plunger positioned at the vascular access point are configured to apply focal pressure at one of a skin level of the subject and the vascular access point of the subject, thus resulting in hemostasis at the vascular access point.

20. The tamponade device as claimed in claim 19, wherein the sheath member adapted to guide the plunger to be positioned at the vascular access point conforms to a guide sheath member.
